(19) United States
(12) Patent Application Publication
(10) Pub. No.: US 2019/0314540 A1
(43) Pub. Date: Oct. 17, 2019

(54) ODOR NEUTRALIZING APPARATUS WITH INTEGRATED NOISE CANCELATION SYSTEM AND METHOD

(71) Applicant: ScentLok Technologies, Inc., Muskegon, MI (US)

(72) Inventor: Nicholas Andrews, Muskegon, MI (US)

(21) Appl. No.: 16/383,076

(22) Filed: Apr. 12, 2019

Related U.S. Application Data
(60) Provisional application No. 62/657,520, filed on Apr. 13, 2018.

(51) Int. Cl. A61L 9/015

(52) U.S. Cl. CPC A61L 9/015; A61L 2209/11; A61L 2209/212; A61L 2209/111; A61L 2209/134

(57) ABSTRACT

A scent neutralizing unit includes a housing, an odor neutralizing gas emitter, and an active noise canceling system. The odor neutralizing gas emitter is coupled to the housing and is configured to provide an odor neutralizing gas that neutralizes odor particles. The active noise canceling system includes a microphone configured to generate a captured signal representing a noise signal, an acoustic transducer configured to emit audio signals, and a controller operatively coupled to the microphone and the acoustic transducer and configured to receive the captured signal from the microphone. The controller is configured to control the acoustic transducer to emit an anti-noise signal based on the captured signal where the anti-noise signal is configured to attenuate the noise signal.

### CROSS-REFERENCE TO RELATED PATENT APPLICATION

This application claims the benefit of and priority to U.S. Provisional Application No. 62/657,520, filed Apr. 13, 2018, which is incorporated herein by reference in its entirety.

### BACKGROUND

Humans produce, expel, facilitate the growth of, or otherwise provide substances that emit a spectrum of odors in the form of odor particles. The rate of production of such substances and, by extension the magnitude of the odors that they emit, increases in hot conditions or when humans participate in exercise. However, even at low magnitudes, the odors produced by such substances can be detected by the acute senses of smell of various animals. Hunters, field biologists, nature photographers, and others rely on remaining undetected by target species (e.g., deer, bears, etc.) during hunting in a natural environment, or in other applications. At greater magnitudes, the odors can be detected by humans and are often considered unpleasant.

Ozone gas is used to neutralize odors. Specifically, upon coming into contact with odor particles, ozone gas chemically reacts with the odor particles, neutralizing them. When used to neutralize odors, ozone gas is typically generated onsite and immediately distributed such that the ozone gas comes into contact with the undesired odor particles. Ozone generation and distribution systems emit audible sounds during operation. Like the odors that the ozone is used to neutralize, such audible sounds are easily detected by acute senses of hearing of humans and/or target species.

### SUMMARY

One embodiment of the present disclosure relates to a scent neutralizing unit including a housing, an odor neutralizing gas emitter, and an active noise canceling system. The odor neutralizing gas emitter is coupled to the housing and is configured to provide an odor neutralizing gas that neutralizes odor particles. The active noise canceling system includes a microphone configured to generate a captured signal representing a noise signal, an acoustic transducer configured to emit audio signals, and a controller operatively coupled to the microphone and the acoustic transducer and configured to receive the captured signal from the microphone. The controller is configured to control the acoustic transducer to emit an anti-noise signal based on the captured signal where the anti-noise signal is configured to attenuate the noise signal.

Another embodiment of the present disclosure relates to a scent neutralizing unit including a housing, an odor neutralizing gas emitter, a fan coupled to the housing, and at least one baffle coupled to the housing. The housing defines an inlet, an outlet, and an internal volume extending between the inlet and the outlet. The odor neutralizing gas emitter is coupled to the housing and is configured to provide an odor neutralizing gas that neutralizes odor particles into the internal volume. The odor neutralizing gas emitter generates an emitter noise signal during operation. The fan is configured to draw air into the internal volume through the inlet and expel a mixture of the air and the odor neutralizing gas from the internal volume through the outlet. The fan generates a fan noise signal during operation. The at least one baffle extends into the internal volume and is configured to attenuate at least one of the emitter noise signal and the fan noise signal.

Another embodiment of the present disclosure relates to a scent neutralizing unit including a housing, an ozone generator, a fan coupled to the housing, at least one baffle coupled to the housing, and an active noise cancellation system. The housing defines an inlet, an outlet, and an internal volume extending between the inlet and the outlet. The ozone generator extends within the housing and is configured to provide ozone gas that neutralizes odor particles. The fan is configured to draw air into the internal volume through the inlet and expel a mixture of the air and the odor neutralizing gas from the internal volume through the outlet. The fan generates a fan noise signal during operation. The at least one baffle extends into the internal volume and is configured to attenuate the fan noise signal. The active noise canceling system includes a microphone configured to generate a captured signal representing the fan noise signal, a speaker configured to produce sound, and a controller operatively coupled to the microphone and the acoustic transducer and configured to receive the captured signal from the microphone. The controller is configured to control the acoustic transducer to emit an anti-noise signal based on the captured signal where the anti-noise signal is configured to attenuate the fan noise signal.

Another embodiment of the present disclosure relates to a method of neutralizing odors including providing, by an odor neutralizing gas emitter, an odor neutralizing gas configured to neutralize odor particles into an internal volume of a housing. The housing defines an inlet and an outlet fluidly coupled to the internal volume. The method further includes moving, by a fan, air into the internal volume through the inlet and expelling, by the fan, a mixture of the air and the odor neutralizing gas through the outlet. The method further includes generating, by at least one of the odor neutralizing gas emitter and the fan, a noise signal and emitting, by an acoustic transducer, an anti-noise signal configured to attenuate the noise signal generated by the at least one of the odor neutralizing gas emitter and the fan.

Alternative exemplary embodiments relate to other features and combinations of features as may be generally recited in the claims.

### BRIEF DESCRIPTION OF THE FIGURES

The disclosure will become more fully understood from the following detailed description, taken in conjunction with the accompanying figures, in which:

FIG. 1 is a front view of a scent neutralizing module, according to an exemplary embodiment;

FIG. 2 is a rear view of the scent neutralizing module of FIG. 1;

FIG. 3 is a top view of the scent neutralizing module of FIG. 1;

FIG. 4 is a bottom view of the scent neutralizing module of FIG. 1;

FIG. 5 is a front exploded view of the scent neutralizing module of FIG. 1;

FIG. 6 is a block diagram of a control system of the scent neutralizing module of FIG. 1;

FIG. 7 is another front view of the scent neutralizing module of FIG. 1; and

FIG. 8 is a schematic view of a Helmholtz resonator of the scent neutralizing module of FIG. 1.

### DETAILED DESCRIPTION

Before turning to the figures, which illustrate the exemplary embodiments in detail, it should be understood that the present application is not limited to the details or methodology set forth in the description or illustrated in the figures. It should also be understood that the terminology is for the purpose of description only and should not be regarded as limiting.

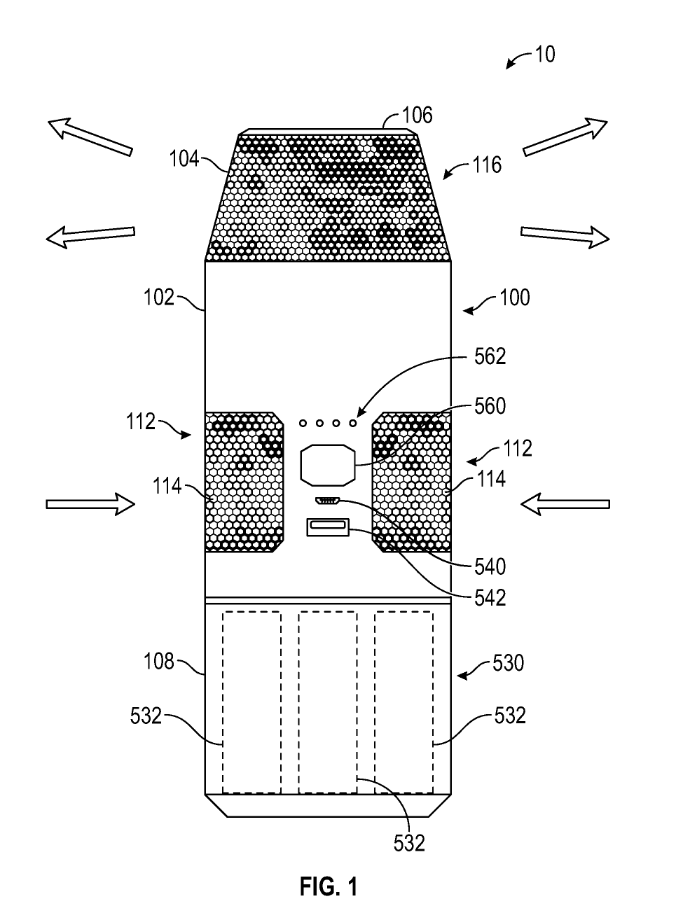

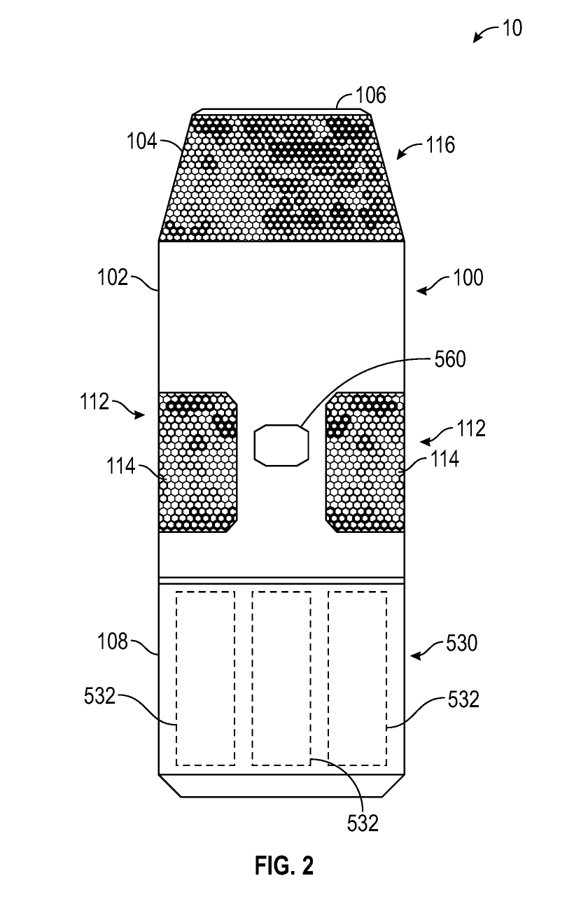

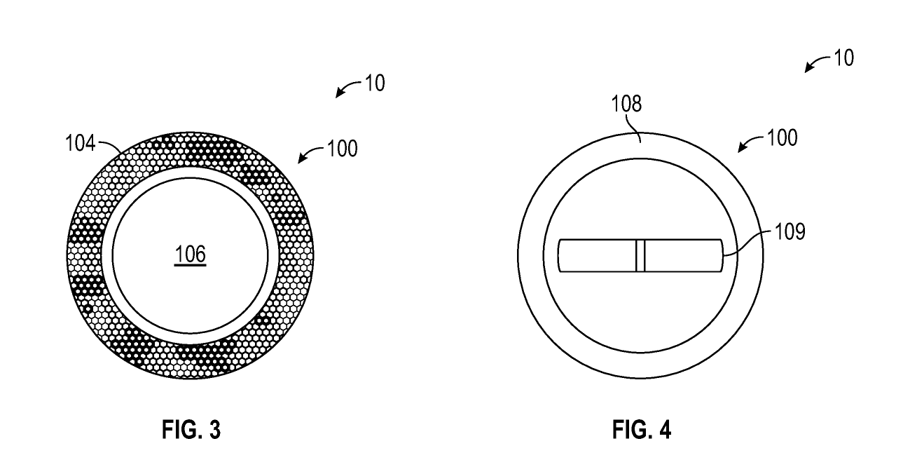

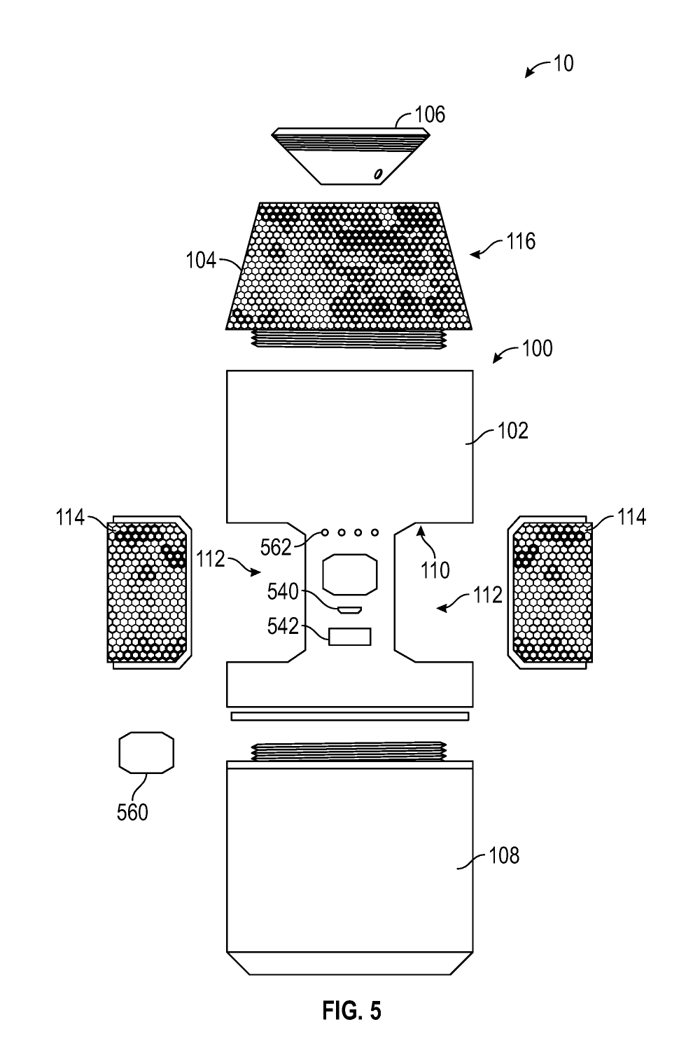

Referring to FIGS. 1-5, an odor or scent neutralizing unit, shown as unit 10, includes a chassis or frame, shown as housing 100. The unit 10 is configured to provide neutralizing gas to neutralize odors and odor causing substances in the surrounding environment. The unit 10 is additionally configured to attenuate undesired sounds or noises that originate in the unit 10 or in the surrounding environment. As shown, the housing 100 is primarily cylindrical and extends about a vertical central axis. However, the housing 100 may be otherwise shaped, for example, the cross-sectional shape can be square, rectangular, arcuate or a polygon. The housing 100 includes a main housing member, shown as main body 102. A top end portion of the main body 102 is coupled to a second housing member, shown as grate 104. A third housing member or deflector, shown as dispersion cone 106, is coupled to a top end portion of the grate 104. A fourth housing member, shown as base member 108, is selectively coupled (e.g., with a threaded connection, with a set of interlocking protrusions and corresponding recesses, etc.) to the bottom end portion of the main body 102. The base member 108 defines an attachment point 109 positioned on a bottom side of the housing 100. The attachment point 109 includes a depression formed in base member 108 and a bar extending across the depression. The attachment point facilitates attaching a carabiner or lanyard to the unit 10. In some embodiments, the housing 100 is approximately 7.75 inches tall and 2.75 inches in diameter.

The individual components of the housing 100 collectively define an internal volume 110. The main body 102 further defines a pair of apertures, shown as inlets 112, each covered by a mesh member or screen, shown as grate 114, coupled to the main body 102. The grates 114 are positioned to extend across the entirety of each inlet 112 and each define a series of apertures that are smaller than the inlets 112. The grate 104 defines a series of apertures, shown as outlets 116. The outlets 116 may be similar in size to the apertures of the grates 114 and the apertures of the grates 114 and the outlets 116 are sized to prevent debris from entering the internal volume 110 while still allowing air to pass freely therethrough. In some embodiments, the grate 104 and the grates 114 are configured to act as filters to prevent dust from entering the internal volume 110. Together the inlets 112, the internal volume 110, and the outlets 116 form a flow path for air flow through the unit 10.

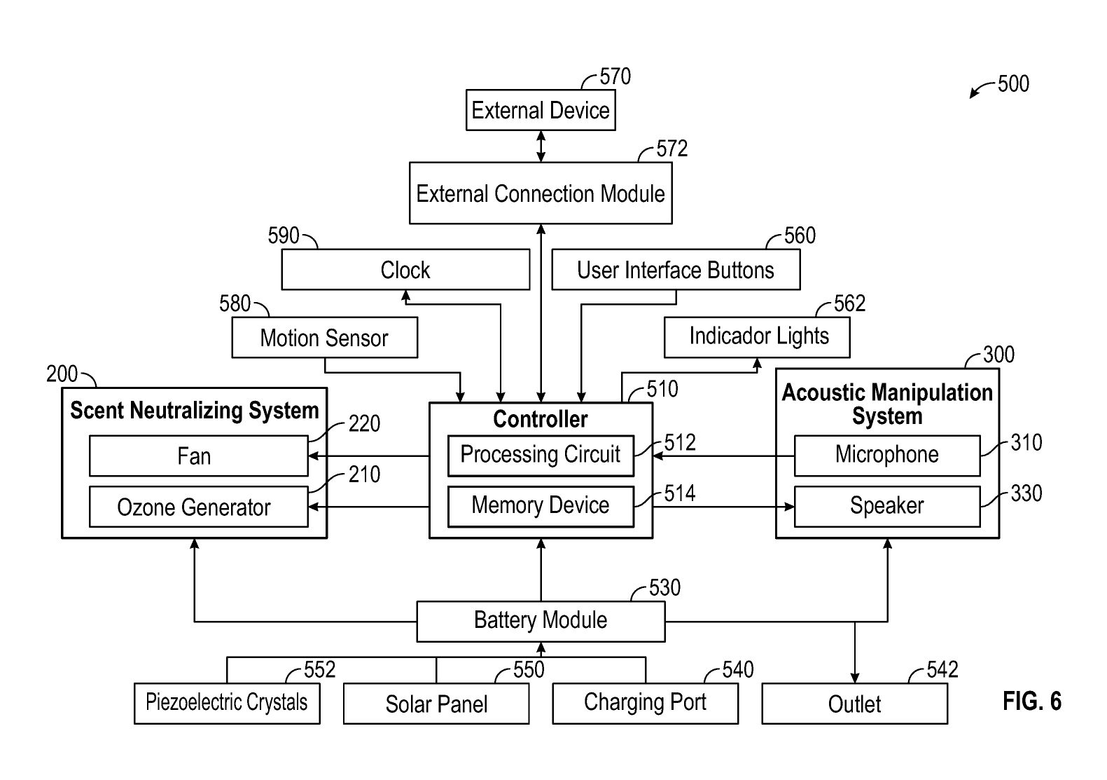

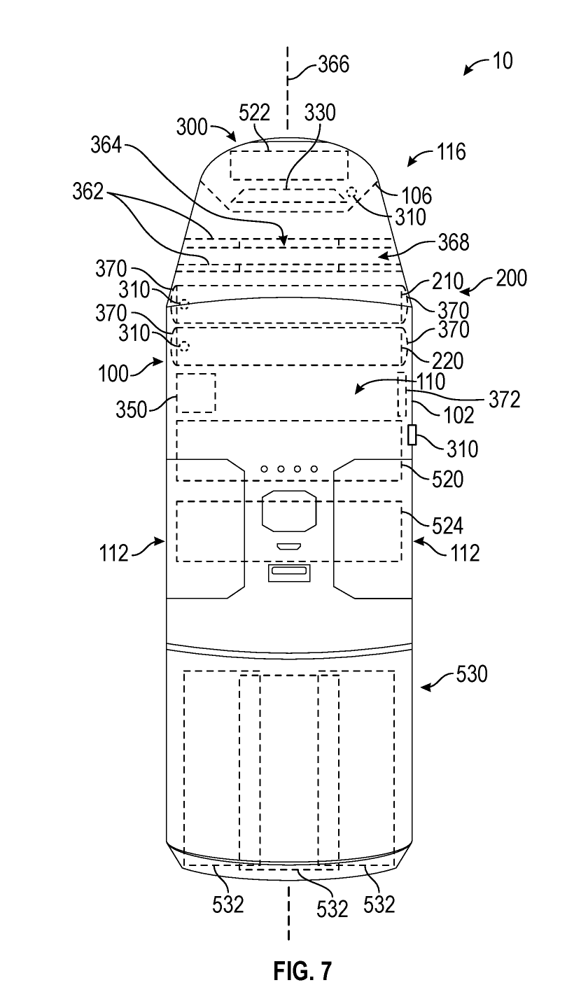

Referring to FIGS. 6 and 7, the unit 10 further includes an odor or scent neutralizing system 200. The scent neutralizing system 200 is configured to release odor neutralizing gas (e.g., ozone, perfume, etc.) that reacts with, destroys, disguises, neutralizes, and/or otherwise modifies odor-emitting substances, odor particles, bacteria, dust mites, and/or other undesirable substances or organisms. After engaging the scent neutralizing system 200, the odors may no longer be detectable by the sense of smell of a human or animal. By way of example, the odor particles may be produced by living beings such as humans, animals, plants, and bacteria (e.g., from sweat, from skin or hair oils, from decomposing material, etc.) or from other sources, such as chemical reactions (e.g., fire, cooking, etc.). The scent neutralizing system 200 may be utilized in order to make a user more difficult for an animal with an acute sense of smell to detect. The scent neutralizing system 200 may be utilized to neutralize odors that a user finds displeasing in their home or vehicle, or be used to eliminate or mask the scent of a human so that when the human enters the field for hunting, it is less likely that the wildlife will detect the human by the sense of smell. By way of example, the unit 10 may be placed inside a vehicle, a gym bag, and/or a hunting bag full of clothes and/or equipment that has absorbed sweat or other materials that emit odor particles. By way of another example, the unit 10 may be placed in a room or container that is filled with trash that emits undesirable odor particles.

The scent neutralizing system 200 includes an odor neutralizing gas emitter, shown as ozone generator 210. As shown in FIG. 7, the ozone generator 210 is coupled to the housing 100 and positioned within the internal volume 110 such that the ozone generator 210 emits odor neutralizing gas into the internal volume 110. The ozone generator 210 is configured to consume electrical energy and generate and emit a supply of gaseous ozone (i.e., 03) that acts as the odor neutralizing gas. In some embodiments, the ozone generator 210 is configured to output approximately 400 milligrams of ozone per hour of continuous operation. Ozone is known to neutralize odor particles, odor-causing bacteria, dust mites, and other undesirable substances and organisms. The ozone generator 210 is configured to convert diatomic oxygen (i.e., 02) from air into ozone. In some embodiments, the ozone generator 210 draws in air from within the internal volume 110. In other embodiments, the ozone generator 210 draws in air from outside of the housing 100 (e.g., through a passage that passes through the housing 100). The ozone generator 210 may be configured to utilize a known method of converting diatomic oxygen into ozone (e.g., corona discharge, ultraviolet radiation, electrolysis, etc.).

In other embodiments, the scent neutralizing system 200 additionally or alternatively includes another type of odor neutralizing gas emitter that is configured to supply odor neutralizing gas. The odor neutralizing gas may be any type of gas or aerosol (e.g., fine particles of liquid dispersed throughout air) capable of reacting with, destroying, disguising, neutralizing, or otherwise modifying odor particles, odor-emitting substances, bacteria, dust mites, and/or other undesirable substances or organisms. By way of a first example, the odor neutralizing system 200 may include an air ionizer configured to electrically charge (i.e., ionize) air molecules, producing ions that remove undesirable substances from the air. In such an example, the odor neutralizing gas may include ionized air. By way of another example, the odor neutralizing system 200 may provide a pressurized supply of perfume or another type of scented substance that disguises odor molecules present in the air. In such an example, the odor neutralizing system 200 may include one or more storage containers configured to hold a concentrated supply of the perfume or scented substance that is later mixed with air to produce the odor neutralizing gas. In some such examples, the perfume is pressurized, and the odor neutralizing system 200 includes an electrically actuated valve that selectively releases the pressurized perfume from the storage container. By way of another example, the odor neutralizing system 200 may be configured to provide a supply of odor oxidizer (e.g., aerosolized odor oxidizer). In some embodiments, the odor neutralizing system 200 includes a filter made from a filter material. The filter material may include activated carbon, zeolite, nano materials, or another type of filter material. The filter may be configured to adsorb or absorb undesirable substances or organisms from the air that passes through the internal volume 110.

Referring again to FIGS. 6 and 7, the odor neutralizing system 200 further includes an air mover, such as a fan or turbine, shown as fan 220. The fan 220 is positioned within the internal volume 110, is coupled to the housing 100, and includes a series of blades adapted to move the adjacent air. The fan 220 is configured to consume electrical energy and rotate such that the blades draw in gas (e.g., air) from upstream of the fan 220 and expel the gas downstream of the fan 220. In some embodiments, as shown in FIG. 7, the fan 220 is positioned upstream of the ozone generator 210. In other embodiments, the fan 220 is positioned downstream of the ozone generator 210. As indicated with arrows in FIG. 1, during operation, the fan 220 generates an air flow that passes from the inlets 112, through the internal volume 110, and out through the outlets 116. Accordingly, air that is drawn in through the inlet 112 mixes with neutralizing gas from the ozone generator 210 in the internal volume 110, and the resultant mixture of air and odor neutralizing gas is expelled from the unit 10 through the outlets 116.

The dispersion cone 106 is configured to facilitate dispersing the mixture of air and neutralizing gas evenly around the unit 10. As shown in FIGS. 1 and 7, air is expelled in a substantially vertical direction from the fan 220, but the outlets 116 are positioned such that the mixture of air and neutralizing gas exits the unit 10 in a primarily lateral direction. The dispersion cone 106 is positioned along the flow path immediately adjacent the outlets 116. The dispersion cone 106 has a surface that is angled relative to the outlets 116 and the fan 220. Specifically, the dispersion cone 106 is substantially conical (e.g., frustoconical, having at least one conical surface, etc.) and centered about the central axis of the housing 100. Accordingly, some of the mixture of air and neutralizing gas contacts the angled surface of the dispersion cone 106 and is redirected laterally to facilitate flow out of the outlets 116. In other embodiments, the dispersion cone 106 has an otherwise angled shape that similarly redirects the flow. By way of example, the dispersion cone 106 may be shaped as a semisphere, a cone, a pyramid, or a tetrahedron.

The unit 10 further includes an acoustic emission system or noise cancellation system, shown as acoustic manipulation system 300. The acoustic manipulation system 300 is configured to attenuate, neutralize, or otherwise reduce the magnitude or presence of undesired noise or sounds (i.e., noise signals). By way of example, the acoustic manipulation system 300 may lower sound pressure levels (e.g., decibels), sound power levels, sound intensity levels, or sound frequency. The acoustic manipulation system 300 may attenuate noise signals originating within the unit 10 and/or noise signals originating within the surrounding environment. The acoustic manipulation system 300 may perform active and/or passive noise cancellation.

In active noise cancellation, the acoustic manipulation system 300 utilizes energy (e.g., electrical energy) to generate, emit, or produce an anti-noise signal (e.g., an audio signal) tuned to attenuate a noise signal. In some embodiments, the acoustic manipulation system observes noise signals and determines the characteristics (e.g., frequencies, loudness, etc.) of the noise signals. In response, the acoustic manipulation system 300 provides an anti-noise signal tuned to attenuate the noise signal of the determined characteristics. In some embodiments, the anti-noise signal has a similar frequency to the corresponding noise signal, but the phases of the sound waves are offset from one another, resulting in destructive interference that reduces the volume (i.e., the loudness) of the noise signal. In other embodiments, the unit 10 does not directly observe the noise signal. Instead, the unit 10 may produce an anti-noise signal that attenuates noise signals within a predetermined frequency range. By way of example, if the characteristics of a noise signal are known to be relatively constant over time (e.g., the sound of the fan 220, etc.), an acoustic transducer (e.g., the speaker 330) may be tuned to produce an anti-noise signal corresponding to those characteristics without direct observation of those noise signals.

In passive noise cancellation, the acoustic manipulation system 300 attenuates the noise signal without actively observing the noise signal and/or without requiring a direct energy input (e.g., without energy from a battery or from a user). By way of example, the unit 10 may be configured to muffle or absorb noise signals prior to the noise signals exiting the unit 10. By way of another example, if the characteristics of the noise signal are known to be relatively constant over time (e.g., the sound of the fan 220, etc.), a resonator may be turned to produce an anti-noise signal tuned to those characteristics without requiring a direct energy input from a battery.

The acoustic manipulation system 300 is advantageous in multiple different situations. By way of example, it can be advantageous for scientific research purposes to attenuate or eliminate any sounds that do not occur in the surrounding natural environment to avoid alerting animals with an acute sense of hearing. While the scent neutralizing system 200 operates in the normal operating position of generating ozone to neutralize odors that may alert the animals, the ozone generator 210 and the fan 220 may generate emitter noise signals and fan noise signals, respectively, (e.g., from rotating motors or gears, electrical whine or other electrical noise, vibration of components within the unit 10, from turbulent air movement, etc.) that may alert the animals, reducing the effectiveness of the scent neutralizing system 200. Additionally, noise signals produced by a user or the user's equipment outside of the unit 10 (e.g., fabric rubbing, breathing, noises from movement of equipment, squeaking of a treestand, etc.) may alert the animals. The acoustic manipulation system 300 is configured to attenuate or eliminate such noise signals, reducing the likelihood that an animal would be alerted. The acoustic manipulation system 300 may additionally or alternatively attenuate or eliminate noises that are annoying or otherwise undesirable to humans, such as electrical whine or other repetitive sounds. As is evident from this disclosure, the odor neutralizing unit 10 is adapted for use by a wide variety of individuals including scientists, biologists, naturalists, hunters, and home owners in a wide variety of locations including in the field, inside a vehicle, cabin, home, or tent, or virtually any other location in which elimination of both odor and minimization of sound are desirable.

The acoustic manipulation system 300 may attenuate noise signals with the goal of reducing the sound pressure of the noise signals as much as possible [e.g., to 0 dB (i.e., no sound)]. In some embodiments, the acoustic manipulation system 300 is configured to reduce noise signals of over 70 dB (e.g., the noise level of a busy street) to a sound pressure level below 30 dB (e.g., a noise level of a soft whisper). Because decibels are measured on a logarithmic scale, each reduction of 10 dB halves the volume of the noise signal. A reduction of 1 dB may be the smallest audible change.

The acoustic manipulation system 300 may be configured to attenuate noise signals within specific frequency ranges. Passive noise cancellation devices and techniques may be used to attenuate noise signals above approximately 10 kHz. Active noise cancellation techniques may be used to reduce attenuate noise signals below approximately 1 kHz. Certain creatures are capable of detecting sounds within different frequency ranges. Humans may be able to hear sounds approximately between 20 Hz and 20 kHz. Deer may be able to hear sounds approximately between 20 Hz and 30 kHz. Dogs may be able to hear sounds approximately between 60 Hz and 50 kHz. Cats may be able to hear sounds approximately between 50 Hz and 80 kHz. The acoustic manipulation system 300 may be configured to attenuate noise signals within any of these ranges. By way of example, it may be beneficial to attenuate noise signals within the frequency range that can be detected by humans to improve the quality of life of a user (e.g., by eliminating annoying sounds, by attenuating loud sounds, etc.). In a scientific research scenario, it may be beneficial to attenuate noise signals within the frequency range that can be detected by deer or other animals, as a reduction in noise signals may reduce the likelihood that the animal will detect a biologist or naturalist. In some embodiments, the acoustic manipulation system 300 is configured to attenuate noise signals approximately between 20 kHz and 30 kHz. Such noise signals may be detectable by deer, but not by humans. Accordingly, such noise signals may alert deer to the presence of the unit 10 without any indication to a user. By way of another example, in a scientific scenario, it may be beneficial to attenuate noise signals within the frequency range that can be detected by dogs, cats, or other domesticated animals, as a reaction of a first animal (e.g., a dog) to a sound may alert a second animal (e.g., deer), even if the second animal is not capable of detecting the sound directly.

Referring to FIGS. 6 and 7, the acoustic manipulation system 300 includes one or more sound receiving devices or sound recording devices, shown as microphones 310. The microphones 310 are configured to receive a vibration, sound, or noise (i.e., an audio signal) and, in response, the microphones 310 are configured to generate and output a captured signal (e.g., an electrical or data signal) that corresponds to the received audio signal. The microphone 310 may be directional (e.g., configured to be more sensitive to audio signals traveling toward the unit 10 in a particular direction) or omnidirectional (e.g., configured to receive audio signals from all directions). A directional microphone may be less sensitive to audio signals coming from certain directions or may avoid receiving audio signals from certain directions entirely. The directional microphone may be more sensitive to audio signals traveling toward the microphone 310 along a first axis (e.g., aligned with a specific source of audio signals) than to audio signals traveling toward the microphone 310 along a second axis angularly offset (e.g., 15 degrees, 90 degrees, 180 degrees, etc.) from the first axis. By way of example, a directional microphone may be oriented towards (e.g., substantially aligned with) a specific source of audio signals (e.g., a naturalist, the fan 220, etc.). Accordingly, the exterior of the housing 100 may be marked (e.g., with a colored ring around the microphone 310, with the shape of a feature of the housing 100) to facilitate aligning a directional microphone with a source of noise signals. In contrast, an omnidirectional microphone may be used to detect ambient audio signals, such as the natural noises of the outdoors.

The locations of the microphones 310 within the unit 10 may be selected to specifically isolate, magnify, or otherwise receive audio signals from a certain source. By way of example, one or more microphones 310 may be placed near components of the scent neutralizing system 200 (e.g., the ozone generator 210, the fan 220, etc.) such that the microphones 310 receive noise signals generated during operation of the scent neutralizing system 200. As shown in FIG. 7, a pair of microphones 310 are positioned adjacent the ozone generator 210 and the fan 220, respectively. Microphones 310 may additionally be placed near components that are prone to emitting electrical noises, such as power supply components. By way of another example, one or more microphones 310 may be placed near one of the inlets 112 or one of the outlets 116 to facilitate receiving noise signals generated by the flow of air and/or neutralizing gas. As shown in FIG. 7, a microphone 310 is positioned within the dispersion cone 106 adjacent the outlets 116. Accordingly, the dispersion cone 106 extends between the microphone 310 and the internal volume 110. The microphone 310 may receive sounds through dispersion cone 106, or the dispersion cone 106 may define an aperture through which the microphone 310 is fluidly coupled to the internal volume 110. By way of another example, one or more microphones 310 may be positioned on or near the exterior of the housing 100. Such a positioning may facilitate identifying both (a) noise signals leaving the unit 10 and (b) noise signals moving toward the unit 10 from an outside source (e.g., a biologist, a treestand, etc.).

Referring again to FIGS. 6 and 7, the acoustic manipulation system 300 further includes an acoustic transducer, shown as speaker 330. The speaker 330 is configured to receive a driving signal (e.g., an electrical or data signal) from a processor and, in response, generate and output an audio signal (e.g., an anti-noise signal) that corresponds to the received driving signal. The speaker 330 is positioned within the dispersion cone 106 and coupled to the housing 100. Accordingly, the dispersion cone 106 extends between the speaker 330 and the internal volume 110. The speaker 330 is oriented such that the speaker emits audio signals away from the internal volume. In some embodiments, the acoustic manipulation system 300 includes multiple speakers 330. In some embodiments, the speaker 330 is removable, and the unit 10 includes multiple receptacles configured to receive the speaker 330 such that the speaker 330 can be rearranged to emit audio signals in different directions depending on the application. By way of example, the speakers 330 may be arranged to emit audio signals toward a user or toward a specific object in the environment. In some embodiments, the location of the speaker 330 is marked on the housing 100 (e.g., with a colored ring around the speaker 330, with the shape of a feature of the housing 100) to facilitate orienting the unit 10 such that the speaker 330 emits audio signals in a desired direction. In some embodiments, the unit 10 includes multiple speakers 330, each configured to emit audio signals within a different frequency range. Accordingly, the speakers 330 may have different physical sizes. This type of arrangement facilitates emitting audio signals across a frequency range broader than that of a single speaker 330.

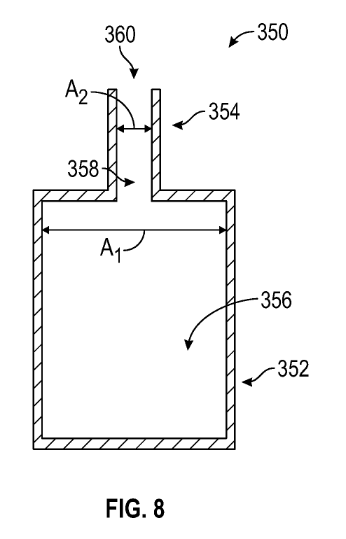

Referring to FIGS. 7 and 8, the acoustic manipulation system 300 further includes a passive noise canceling or attenuation device or resonator, shown as Helmholtz resonator 350. The Helmholtz resonator 350 has a body portion 352 adjacent a neck portion 354 thereby defining a resonator volume or cavity 356 and a resonator passage or passage 358, respectively. The cavity 356 is fluidly coupled to the passage 358, and the passage 358 terminates with an inlet 360 that is fluidly coupled to the internal volume 110. The cavity 356 has a first cross sectional area A1, and the passage 358 has a second cross sectional area A2 that is less than A1. When the fan 220 is operating, air flowing through the internal volume 110 moves across an opening of the passage 358, causing the Helmholtz resonator 350 to emit an audio signal of a predetermined frequency. The predetermined frequency is related to the geometry of the Helmholtz resonator 350 (e.g., the length of the passage 358, the volume of the cavity 356, etc.). Accordingly, the Helmholtz resonator 350 is configured (e.g., sized) such that the audio signal emitted by the Helmholtz resonator 350 is an anti-noise signal that neutralizes one or more particular noise signals. By way of example, during operation, the fan 220 may emit a noise signal having substantially constant characteristics, and the Helmholtz resonator 350 may be configured to emit an anti-noise signal that neutralizes that noise signal.

As shown in FIG. 8, the acoustic manipulation system 300 further includes a series of panels, shown as baffles 362, that are configured to passively attenuate noise signals. The baffles 362 are coupled to the housing 100 and extend radially inward from the walls of the housing 100 (e.g., the main body 102, the grate 104, etc.). The baffles 362 define a series of apertures, shown as ventilation apertures 364, through which air and odor neutralizing gas may travel. In some embodiments, the ventilation apertures 364 are approximately centered about a central longitudinal axis 366 of the housing 100. In some embodiments, the baffles 362 are substantially planar, extending substantially perpendicular to the longitudinal axis 366. In some embodiments, the baffles 362 are disc-shaped. In other embodiments, the baffles 362 extend at an angle (e.g., 45 degrees, etc.) relative to the longitudinal axis 366. By way of example, the baffles 362 may be substantially frustoconical. Each pair of adjacent baffles 362 defines a duct, chamber, or passage, shown as duct 368, therebetween. The ducts 368 are in fluid communication with the inlet 112 and the outlet 116 such that air and/or odor neutralizing gas can flow across and/or through the ducts 368. The baffles 362 and/or other surfaces of the unit 10 direct sound waves into the ducts 368, where the sound energy can be absorbed and dissipated (e.g., converted to thermal energy). In other embodiments, the ducts 368 direct the sound waves into another volume (e.g., defined by the housing 100) where the sound energy is absorbed. The baffles 362 also reduce turbulence of the air and neutralizing gas flowing through the housing 100, thereby reducing the noise signals produced by flowing gas (e.g., by directing gas into the ducts 368).

The baffles 362 and/or other surfaces of the unit 10 may be formed with ridges, pores, or other surface features that facilitate absorption of sound energy and/or conversion of sound energy to thermal energy. In operation, the baffles 362 passively absorb vibration, attenuating noise signals that come into contact with the baffles 362. Based on the characteristics of the noise signals that will be attenuated, the baffles 362 may be configured to have a specific absorption (e.g., an amount of sound energy that is absorbed for a given noise signal), surface mass (e.g., an amount of pores, ridges, or other features that convert sound energy to thermal energy, a ratio of surface area to mass), and/or ability to absorb sounds having certain frequencies.

The acoustic manipulation system 300 may further include one or more diffusers, screens, or grates (e.g., the grate 104) that reduce turbulence of the air and neutralizing gas flowing through the housing 100, thereby passively reducing the noise signals produced by flowing gas. The dispersion cone 106 acts as a passive noise cancellation device, reducing turbulence by smoothly redirecting flow through the internal volume 110 toward the outlet 116. Referring to FIG. 7, the unit 10 includes anti-vibration isolators, shown as compliant blocks 370, that couple the ozone generator 210 and the fan 220 to the housing 100. The compliant blocks 370 may be made from a compliant material, such as rubber, plastic, or cork. The compliant blocks 370 extend between the ozone generator 210 and the housing 100 and between the fan 220 and the housing 100, preventing direct contact between the ozone generator 210 or the fan 220 and the housing 100. The compliant blocks 370 reduce the transfer of vibration from the ozone generator 210 and the fan 220 to the housing 100 and the generation of noise signals caused by contact between a vibrating component and a stationary component. In some embodiments, the housing 100 includes an insulative material (e.g., noise dampening foam, etc.) that attenuates audio signals passing therethrough. By way of example, the housing 100 may include a plastic outer shell with a layer of noise dampening foam 372 extending across an inside surface of the housing 100.

Referring again to FIG. 6, the unit 10 includes a control system 500 which includes a controller 510 that is configured to control operation of the unit 10. The controller 510 is operatively coupled to and configured to control the ozone generator 210 and the fan 220. The controller 510 is operatively coupled to the microphones 310 and configured to receive the captured signal. The controller is operatively coupled to the speaker 330 and configured to provide a driving signal to control the speaker 330.

The controller 510 may be implemented as a general-purpose processor, an application specific integrated circuit (ASIC), one or more field programmable gate arrays (FPGAs), a digital-signal-processor (DSP), circuits containing one or more processing components, circuitry for supporting a microprocessor, a group of processing components, or other suitable electronic processing components. According to the exemplary embodiment shown in FIG. 6, controller 510 includes a processing circuit 512 and a memory device 514. The processing circuit 512 may include an ASIC, one or more FPGAs, a DSP, circuits containing one or more processing components, circuitry for supporting a microprocessor, a group of processing components, or other suitable electronic processing components. In some embodiments, the processing circuit 512 is configured to execute computer code stored in the memory device 514 to facilitate the activities described herein. The memory device 514 may be any volatile or non-volatile computer-readable storage medium capable of storing data or computer code relating to the activities described herein. According to an exemplary embodiment, the memory device 514 includes computer code modules (e.g., executable code, object code, source code, script code, machine code, etc.) configured for execution by the processing circuit 512. The memory device 514 includes various actuation profiles corresponding to modes of operation (e.g., for ozone generator 210, for fan 220, for speaker 330, etc.), according to an exemplary embodiment. In some embodiments, controller 510 may represent a collection of processing devices (e.g., separate control modules within the unit 10, etc.). By way of example, FIG. 7 shows the controller 510 as including three separate printed circuit boards. A scent neutralizing module 520 controls the scent neutralizing system 200. A noise cancellation module 522 controls operation of the acoustic manipulation system 300. A power management module 524 controls the distribution of electrical power to various components of the unit 10. In such cases, the processing circuit 512 represents the collective processors of the devices, and memory device 514 represents the collective storage devices of the devices.

Referring to FIGS. 1, 6, and 7, the unit 10 further includes an energy storage device, shown as battery module 530. The battery module 530 is configured to supply electrical energy to power the components of the unit 10 (e.g., the ozone generator 210, the fan 220, the speaker 330, the controller 510, etc.). The battery module 530 may be directly electrically coupled to each component, or the battery module 530 may be indirectly coupled to each component (e.g., through the controller 510). The battery module 530 is positioned within and coupled to the base member 108 of the housing 100 such that the battery module 530 is selectively removable from the unit 10. By way of example, the battery module 530 may be removed and recharged separately when depleted, or the battery module 530 may be replaced with a second battery module 530 that is fully charged. In some embodiments, the base member 108 and the battery module 530 are removable together as an assembly. The battery module 530 includes one or more battery cells 532. The battery cells 532 may be rechargeable. In some embodiments, the battery module 530 is sized such that the unit 10 can operate for between 4 and 5 hours on a single charge.

Referring to FIGS. 1 and 6, the unit 10 further includes an electrical energy inlet, shown as charging port 540. The charging port 540 is positioned along a front side of the housing 100. The charging port 540 is configured to receive an electrical connector and couple the unit 10 to an external power source (e.g., another battery, a generator, the power grid, etc.). The unit 10 may use electrical energy from the external power source to power various functions of the unit 10, to charge the battery module 530, or both simultaneously. Accordingly, the unit 10 may be configured to operate without the battery module 530 when receiving electrical energy through the charging port 540. The housing 100 further defines an electrical energy outlet, shown as outlet 542. The outlet 542 is also positioned along the front side of the housing 100. The outlet 542 is configured to receive an electrical connector to electrically couple the battery module 530 to an external device, such as a mobile phone or flashlight, thereby powering the external device. The charging port 540 and the outlet 542 may be standardized types of ports to facilitate connection to other devices. By way of example, as shown in FIG. 1, the charging port 540 is a female micro universal serial bus (USB) port, and the outlet 542 is a female USB type A port.

Referring to FIG. 6, in some embodiments, the unit 10 includes one or more other types of onboard power sources. In some embodiments, the unit 10 includes a photovoltaic panel or array of photovoltaic panels, shown as solar panel 550. The solar panel 550 is configured to receive light (e.g., sunlight) incident on an exterior surface of the solar panel 550 and generate electrical energy to power the unit 10 and/or charge the battery module 530. The solar panel 550 may be coupled to an exterior surface of the housing 100 such that the solar panel 550 is exposed to light from the environment surrounding the unit 10. In some embodiments, the unit 10 includes one or more piezoelectric crystal assemblies, shown as piezoelectric crystals 552. The piezoelectric crystal 552 includes a crystalline substance that is configured to generate a voltage in response to mechanical stresses (e.g., from deformation, from vibration, etc.). The piezoelectric crystals 552 are coupled to the housing 100 such that the piezoelectric crystals 552 are exposed to vibrations and generate electrical energy to power the unit 10 and/or charge the battery module 530. By way of example, the vibrations may originate from movement of the unit 10 (e.g., when the unit 10 is placed in a bag and carried or otherwise moved such that the piezoelectric crystals 552 are shaken). By way of another example, the vibrations may originate from movement of a component within the unit 10 (e.g., a piezoelectric crystal 552 may be mounted to a shroud of the fan 220 such that the piezoelectric crystal 552 vibrates due to operation of the fan). By way of another example, the vibrations may be audio signals (e.g., noise signals from the fan 220, audio signals from the speaker 330, noise signals from the environment surrounding the unit 10 etc.) incident on the piezoelectric crystals 552.

Referring to FIGS. 1, 2, and 6, the unit 10 includes a pair of user interface devices, shown as user interface buttons 560. The user interface buttons 560 each extend through and are coupled to the main body 102, one on the front side of the housing 100 and one on a back side of the housing 100. The user interface buttons 560 are each operatively coupled to the controller 510 and configured to provide a user interface signal to the controller 510 when depressed. The controller 510 is configured to control operation of the unit 10 based on the user interface signals from the user interface buttons 560. A user may provide different commands to the controller 510 by varying: which user interface button 560 or user interface buttons 560 are depressed, the length of time that each user interface button 560 is depressed, and the length of time between depressions of the user interface button 560. In other embodiments, the unit 10 includes more or fewer user interface buttons 560. In yet other embodiments, another type of user interface is used to control the operation of the unit 10. By way of example, the unit 10 may include a toggle switch that is selectively repositionable between an “on” position in which the toggle switch electrically couples the battery module 530 and/or the charging port 540 to the other components of the unit 10 and an “off” position in which the toggle switch electrically decouples the battery module 530 and/or the charging port 540 from the other components of the unit 10. By way of another example, the unit 10 may include knobs, touch screens, joysticks, or other user interface devices.

Referring to FIGS. 1 and 6, the unit 10 further includes a visual indicator, shown as indicator lights 562. The unit 10 includes four indicator lights 562 coupled to the housing 100 and operatively coupled to the controller 510. The indicator lights 562 are arranged in a row and visible from outside of the housing 100. The indicator lights 562 may be configured to emit light of fixed or variable colors and intensities. The controller 510 may be configured to control each indicator light 562 individually. Accordingly, the controller 510 may be configured to control the color of the emitted light, the intensity of the emitted light, and when each light is illuminated (e.g., a duration, a relative timing between illuminations of each indicator light 562, etc.) in order to communicate information to a user. In other embodiments, the unit 10 includes other types of visual indicators, such as dot matrix displays or liquid crystal displays.

Referring to FIG. 6, in some embodiments, the unit 10 is configured to communicate with an external device 570. The external device 570 may be a portable device such as smartphone or tablet, a remote control, a computer, a server, or another type of device configured to send commands and/or receive information. The unit 10 includes a controller, shown as external connection module 572, that is configured to facilitate communication between the external device 570 and the controller 510. The external connection module 572 and the external device 570 may communicate using two-way communication or one-way communication. The external connection module 572 may communicate over a wired or wireless connection. In some embodiments, the external connection module 572 includes a wire or cable that directly connects the controller 510 and the external device 570. In other embodiments, the external connection module 572 is configured to communicate with the external device 570 using infrared light. By way of example, the external device 570 and the external connection module 572 may each include an infrared emitter and an infrared receiver to facilitate two-way communication of commands and information. By way of another example, the external device 570 may include an infrared emitter, and the external connection module 572 may include an infrared receiver to facilitate one-way communication of commands from the external device 570 to the controller 510. In other embodiments, the external device 570 and the external connection module 572 may communicate using Bluetooth, near field communication, radio frequency communication, over a network (e.g., the Internet), or using another type of communication.

Referring again to FIG. 6, in some embodiments, the unit 10 includes a motion detector, shown as motion sensor 580. The motion sensor 580 is coupled to the housing 100 and operatively coupled to the controller 510. The motion sensor 580 is configured to detect movement relative to the unit 10 of objects (e.g., humans, animals, inanimate objects, etc.) near the unit 10 (e.g., within a line of sight of the unit 10, etc.). Upon detecting movement, the motion sensor 580 is configured to provide a motion detection signal to the controller 510 indicating that motion is detected. The motion sensor 580 may additionally or alternatively be configured to detect movement of the unit 10 relative to the environment (e.g., if the unit 10 is picked up or bumped, etc.). The motion sensor 580 may include a passive infrared sensor, a microwave sensor, an ultrasonic sensor, a video camera, an accelerometer, or another type of sensor configured to detect motion.

Referring again to FIG. 6, in some embodiments, the unit 10 includes a timekeeping device, shown as clock 590, operatively coupled to the controller 510. The clock 590 is configured to track at least one of a current date, a current time, and a time since an event occurred (e.g., a time since the unit 10 was powered on, a time since a button was pushed, etc.). The clock 590 is configured to provide time data related to at least one of the current date, the current time, and a time since the event occurred to the controller 510. As shown in FIG. 7, the clock 590 is positioned onboard the unit 10 (e.g., included as part of the power management module 524 of the controller 510.)

During operation of the unit 10, the controller 510 controls the operation of the scent neutralizing system 200 to neutralize undesirable odors in the vicinity of the unit 10. The controller 510 additionally or alternatively controls the acoustic manipulation system 300 to attenuate or eliminate undesirable noise signals. These noise signals may be produced by the unit 10 or may originate from a source outside the unit 10. The controller 510 may control the scent neutralizing system 200 and the noise cancellation systems to operate independently or simultaneously. In some embodiments, the acoustic manipulation system 300 is automatically activated when the scent neutralizing system 200 is activated to attenuate noise signals produced by the scent neutralizing system 200 during operation.

The controller 510 is configured to control the components of the unit 10 according to various modes of operation based on various inputs (e.g., user inputs provided by a user, inputs from the environment, etc.). Prior to operation of the unit 10, the controller 510 may be configured to operate in an off or standby mode of operation in which the unit 10 consumes a minimal amount of electrical energy (e.g., in which the scent neutralizing system 200 and the acoustic manipulation system 300 do not operate). While in the standby mode of operation, the controller 510 awaits a command from a user to enter an active mode of operation (e.g., a mode of operation in which the scent neutralizing system 200 and/or the acoustic manipulation system 300 operate). Once in an active operation mode, the controller 510 may be configured to change various operating parameters (e.g., how much neutralizing gas is generated by the ozone generator 210, the speed of the fan 220, how the acoustic manipulation system 300 identifies noise signals, what audio signals are emitted by the acoustic manipulation system 300, what information is provided to the user, what external device 570 the unit 10 is connected to, etc.) in response to various inputs.

The unit 10 is configured to respond to multiple types of inputs. The controller 510 may respond to a user depressing the user interface buttons 560 (e.g., based on which user interface buttons 560 are pressed, how long the user interface buttons 560 are pressed, how many times a user interface button 560 is pressed within a predetermined time period, etc.). The controller 510 may respond to the unit 10 receiving electrical energy though the charging port 540 or an external device being connected to the outlet 542.

The controller 510 may be configured to respond to an input from the clock 590. By way of example, the controller 510 may use the time data from the clock 50 to determine whether the controller 510 is operating during a standby time range or an active time range. These time ranges may each contain certain dates, certain times of the day, and/or certain periods since an event has occurred. By way of example, the standby time range may contain a time period in which users are expected to be present near the unit 10, and the active time range may contain time periods during which the user is not expected to be present near the unit 10. By way of another example, the controller 510 may be configured to enter the active time range from the standby time range after a user-specified period of time has elapsed since a user started a timer (e.g., by pressing a button). By way of another example, the controller 510 may be configured to enter the standby time range from the active time range after a predetermined period of time has elapsed since the unit 10 was powered on. The controller 510 may operate in a standby mode of operation during the standby time range and in an active mode during the active time range.

The controller 510 may be configured to respond to an input from the motion sensor 580. By way of example, the controller 510 may be configured to change from a standby mode to an active mode of operation in response to the motion sensor 580 detecting motion. The controller 510 may additionally or alternatively be configured to change from an active mode to a standby mode in response to the motion sensor 580 not detecting motion for a threshold period of time. Accordingly, the unit 10 may be configured to emit neutralizing gas when a user is present and stop emitting neutralizing gas when a user is not present, conserving electrical energy. By way of another example, the controller 510 may be configured to change from an active mode to a standby mode in response to the motion sensor 580 detecting motion. The controller 510 may additionally or alternatively be configured to change from a standby mode to an active mode in response to the motion sensor 580 not detecting motion for a threshold period of time. Accordingly, the unit 10 may be configured to stop emitting neutralizing gas when a user is present and emit neutralizing gas when a user is not present, minimizing contact between the neutralizing gas and the user.

The controller 510 may be configured to receive inputs (e.g., commands, etc.) from the external device 570 through the external connection module. By way of example, the external device 570 may be a smartphone connected to the unit 10 through a Bluetooth connection. The external device 570 may be configured to run an application that provides a graphical user interface (GUI), through which the user may change various settings relating to the operation of the unit 10. In response to the user selecting certain settings, the external device 570 may provide commands to the controller 510 through the external connection module 572. By way of another example, the external device 570 may be a remote control having a series of labeled buttons. In response to a user pressing certain buttons on the remote control, the remote control may emit infrared signals representing commands which are then received by the external connection module 572. Alternatively, such a remote control may communicate with the controller 510 over a wired connection.

The controller 510 may be configured to respond to certain audio signals being received by the microphone 310. The controller 510 may change from a standby mode to an active mode of operation in response to receiving a certain audio signal (e.g., an audio signal of a particular frequency such as that of footsteps, an audio signal having greater than a threshold volume, etc.). The controller 510 may be configured to operate the scent neutralizing system 200 without the acoustic manipulation system 300 emitting anti-noise signals to conceal the scent neutralizing system 200. While the scent neutralizing system 200 operates, the controller 510 may be configured to use the microphone 310 to monitor for the presence of audio signals that would indicate the presence of a human or animal (e.g., footsteps, breathing, speech, mating calls, etc.). Once such a noise is detected, the controller 510 may be configured to control the acoustic manipulation system 300 to emit anti-noise signals that attenuate noise signals emitted by the scent neutralizing system 200.

The controller 510 may additionally or alternatively be configured to respond to voice commands from a user. The controller 510 may be configured to use the microphone 310 to monitor for the presence of audio signals corresponding to human speech. Once such a signal is received, the controller 510 may be configured to operate in a specific manner based on the specific phrase spoken by the user. By way of example, the controller 510 may be configured to change to a standby mode of operation in response to a user speaking the phrase “turn off” By way of another example, the controller 510 may be configured to operate the scent neutralizing system 200 in response to a user speaking the phrase “begin neutralizing.”

The unit 10 may be configured to provide the user with various useful pieces of information. By way of example, the controller 510 may communicate to a user the current mode of operation, a charge level of the battery module 530, whether the scent neutralizing system 200 or the acoustic manipulation system 300 are running, or other types of information. The controller 510 may communicate information to the user using the indicator lights 562. By way of example, the controller 510 may indicate that the unit 10 is in a standby mode of operation by not illuminating any of the indicator lights 562. The controller 510 may indicate that the unit 10 is in a first active mode of operation by repeatedly cycling illumination of each indicator light 562 in a first sequence (e.g., from left to right, etc.). The controller 510 may indicate that the unit 10 is in a second active mode operation by repeatedly cycling illumination of each indicator light 562 in a second sequence (e.g., from left to right then right to left, etc.). Alternatively, the controller may change the color or intensity of each indicator light 562 to communicate information. In some embodiments, the controller 510 communicates information to the user through the external device 570 using the external connection module 572. By way of example, external device 570 may have a screen that provides the information.

While in an active mode of operation, the controller 510 may be configured to operate the scent neutralizing system 200. During operation, the fan 220 consumes electrical energy and rotates, drawing air into the internal volume 110 through the inlet 112. The ozone generator 210 uses electrical energy and diatomic oxygen (e.g., from the air drawn into the internal volume 110 by the fan 220) to generate neutralizing gas. The air from the fan 220 moves around the ozone generator 210, mixing with the neutralizing gas from the ozone generator 210. Positive pressure within the internal volume 110 caused by the fan 220 forces the mixture of air and neutralizing gas out of the internal volume 110 through the outlets 116. The neutralizing gas then disperses in an area around the unit 10, neutralizing odor particles and odor causing substances in the surrounding atmosphere and on other surfaces.

During operation, the controller 510 may vary the speed of the fan 220 and/or the rate at which the neutralizing gas is emitted by the ozone generator 210. By way of example, in some situations, a user may desire a greater level of neutralizing effectiveness (e.g., a greater concentration of neutralizing gas to be distributed within a certain area, an increase in the size of the area in which the neutralizing gas is distributed). The unit 10 may provide such an increase by increasing the speed of the fan 220 (e.g., to cause a greater air pressure within the internal volume 110, thereby distributing the neutralizing gas over a larger area). The unit 10 may provide such an increase by increasing the rate at which neutralizing gas is emitted by the ozone generator 210. However, such operations may consume a greater amount of electrical energy, reducing the battery life of the battery module 530. Alternatively, a user may desire a greater battery life at the cost of reduced neutralizing effectiveness. Accordingly, in response to a command indicating such a desire, the controller 510 may be configured to reduce the speed of the fan 220 and/or reduce the rate at which the ozone generator 210 emits neutralizing gas.

According to an exemplary embodiment, the controller 510 is configured to change between modes of operation in response to a user interacting with the user interface buttons 560. The unit 10 begins in an off or standby mode in which the scent neutralizing system 200 and the acoustic manipulation system 300 are inactive and the controller 510 awaits a user input. When a user depresses one of the user interface buttons 560 for a brief period of time (e.g., less than three seconds, etc.), the controller 510 configures the unit 10 into a regular mode of operation. In the regular mode, the controller 510 runs the fan 220 at a slow speed for increased battery life. When a user depresses both of the user interface buttons 560 simultaneously for a brief period of time, the controller 510 configures the unit 10 into a boost mode of operation. In the boost mode, the controller 510 runs the fans at a high speed for increased odor neutralizing effectiveness. When a user depresses one of the user interface buttons 560 for an extended period of time (e.g., more than three seconds, etc.), the controller 510 configures the unit 10 into a cycle mode of operation. In the cycle mode, the controller 510 alternates between running and not running the ozone generator 210 and the fan 220 for preset periods of time (e.g., half hour periods, etc.). By way of example, a fully charged battery module 530 may be sufficient for approximately five hours of operation while in the regular mode, approximately four hours of operation while in the boost mode, and approximately eight hours of operation while in the cycle mode (e.g., four hours of running the ozone generator 210 and the fan 220 and four hours of not running the ozone generator 210 and the fan 220). While in the regular mode, the boost mode, or the cycle mode, if a user taps one of the user interface buttons 560, the controller 510 configures the unit 10 back into the standby mode.

While in an active mode of operation, the controller 510 may be configured to operate the acoustic manipulation system 300. In some embodiments, the microphones 310 are configured to receive at least one undesired noise signal and generate a captured signal representing the noise signal. The controller 510 uses the captured signal to determine characteristics (e.g., a frequency, etc.) of an anti-noise signal that will attenuate or eliminate the noise signal. The controller 510 then generates a driving signal representing the anti-noise signal. The speaker 330 receives the driving signal and emits the anti-noise signal. The anti-noise signal passes into the air surrounding the unit 10, attenuating or eliminating the noise signal.

In some embodiments, the acoustic manipulation system 300 is configured to attenuate a noise signal originating within the unit 10. By way of example, the noise signal may be emitted by the ozone generator 210, the fan 220, the controller 510, or another component of the unit 10. By way of another example, the noise signal may be generated by air flow. In one such embodiment, the unit 10 includes a microphone 310 that is positioned adjacent the location where the noise signal is generated (e.g., adjacent the fan 220, adjacent the ozone generator 210, etc.). Such positioning may facilitate detecting the noise signal without inadvertently detecting other audio signals. In some embodiments, the microphone 310 is a directional microphone that is oriented toward the location where the noise signal is generated, further improving detection of the noise signal. Using the captured signal from the microphone 310, the controller 510 controls the speaker 330 to emit an anti-noise signal configured to attenuate the noise signal. The speaker 330 may be positioned to emit the anti-noise signal outward (i.e., away from the module) or inward (i.e., into the internal volume 110).

In some embodiments, the acoustic manipulation system 300 is configured to attenuate a noise signal originating outside the unit 10. By way of example, the noise signal may be emitted by a living entity (e.g., a human that is breathing or moving, etc.) or by an inanimate object (e.g., a pair of metal components that rub against one another, etc.). In one such embodiment, the unit 10 includes a microphone 310 that is positioned on the exterior of the housing 100 to facilitate detecting the noise signal. In embodiments where the source of the noise signal is known (e.g., a human seated in a stationary position), the microphone 310 may be a directional microphone that the user orients toward the source of the noise signal. In other embodiments, the microphone 310 may be omnidirectional to facilitate receiving the noise signal from multiple directions. Using a captured signal from the microphone 310 corresponding to the noise signal, the controller 510 controls the speaker 330 to emit an anti-noise signal configured to attenuate the noise signal. The speaker 330 may be positioned to emit the anti-noise signal outward (i.e., away from the module). In embodiments where the source of the noise signal is known, the user may orient the speaker 330 toward the source of the noise signal.

Alternatively, the acoustic manipulation system 300 may be configured to attenuate a noise signal having known characteristics (e.g., a known or predetermined frequency range, etc.). By way of example, during operation, the fan 220 may emit a noise signal having substantially constant characteristics. These characteristics may be predetermined and stored in the memory device 514. By way of another example, the fan 220 may emit a noise signal having characteristics that have a predetermined relationship to the speed of the fan 220. This relationship may be stored in the memory device 214, and the controller 510 may be configured to use a current speed of the fan 220 to determine the characteristics of the noise signal. Accordingly, the controller 510 may be configured to use the predetermined characteristics or the predetermined relationship to determine an anti-noise signal that will attenuate the noise signal. The controller 510 may then control the speaker 330 to emit the anti-noise signal without directly measuring the characteristics of the noise signal (e.g., without using a microphone 310).

In some embodiments, the acoustic manipulation system 300 is configured to receive multiple audio signals, and the controller 510 is configured to determine which of the audio signals are undesired noise signals. In one embodiment, the unit 10 is placed within an environment during a teaching or learning period. During the teaching period, the source of the noise signals (e.g., a human, etc.) is removed, disabled, or neutralized, and one or more microphones 310 receive audio signals that are emitted naturally by the environment. By way of example, the ozone generator 210 and the fan 220 may be disabled (e.g., turned off, deactivated), thereby removing them as sources of noise signals. The controller 510 is configured to categorize these audio signals as background audio signals, and the characteristics of these audio signals (e.g., the corresponding captured signal generated by the controller) are stored in the memory device 514. A working period follows the teaching period. During the working period, the source of the noise signals is introduced into the environment and emits noise signals. By way of example, the ozone generator 210 and/or the fan 220 may be reactivated, and/or the user may be present. The controller 510 is configured to compare the characteristics of the audio signals received during the working period against those received during the teaching period and isolate the noise signals. The controller 510 then controls the speaker 330 to emit anti-noise signals configured to attenuate the noise signals.

In some embodiments where the location of the source of the noise signals is known, the unit 10 includes at least two microphones 310: a directional microphone and an omnidirectional microphone. The directional microphone is configured to be oriented toward the source of the noise signals by the user. The omnidirectional microphone is positioned to receive both background audio signals and the noise signals. Because the directional microphone is oriented toward the source of the noise signals, the noise signals received by the directional microphone are stronger than the noise signals received by the omnidirectional microphone. The controller 510 may be configured to compare the captured signal from the directional microphone against the captured signal from the omnidirectional microphone to determine which audio signals were significantly stronger as measured by the directional microphone than as measured by the omnidirectional microphone. The controller 510 may determine that these stronger audio signals are the noise signals. The controller 510 then controls the speaker 330 to emit anti-noise signals configured to attenuate the noise signals.

In some embodiments, the acoustic manipulation system 300 is configured to attenuate all audio signals within a predetermined frequency range. In other embodiments, the acoustic manipulation system 300 is configured to attenuate all audio signals outside of a predetermined frequency range. By way of example, the acoustic manipulation system 300 may be configured to attenuate all audio signals outside of the range of human hearing (e.g., below 16 Hz or above 20 kHz, etc.). A microphone 310 (e.g., an omnidirectional microphone) may be configured to receive all audio signals within an environment. Using the captured signal from the microphone 310, the controller 510 may be configured to control the speaker 330 to emit anti-noises that attenuate all of the audio signals outside of the predetermined range.

In some embodiments, the acoustic manipulation system 300 is configured to emit audio signals other than anti-noise signals. The audio signals emitted by the acoustic manipulation system 300 may be useful, pleasing to the user, or otherwise desirable. By way of example, the acoustic manipulation system 300 may be configured to emit sounds that attract animals (e.g., the sound of a running deer, the sound of a doe in heat, the sound of rustling leaves, bird calls, etc.). By way of another example, the acoustic manipulation system 300 may emit audio signals that are pleasing to the user (e.g., talk radio, music, etc.). Such noises may be prerecorded and stored onboard the unit 10 (e.g., in the memory device 514), may be generated by the controller 510, or may be provided to the unit 10 by the external device 570. By way of example, music files may be stored on a portable music player or smartphone and provided to the unit 10 through the external connection module 572 (e.g., through Bluetooth communication, through a wired connection, etc.). By way of another example, the controller 510 may access music files stored remotely on a server through an Internet connection.

The unit 10 may be configured to record audio signals (e.g., for later playback by the unit 10, to be provided to an external device 570). By way of example, the unit 10 may be used for hands-free calling. In such an example, the controller 510 may record a user's voice using the microphone 310 and provide a recorded audio signal representing the user's voice to the external device 570 using the external connection module 572. The external device may provide a second recorded audio signal representing a voice of a second person to the controller 510 through the external connection module 572. The controller 510 may then control the speaker 330 to emit an audio signal corresponding to the second recorded audio signal.

The acoustic manipulation system 300 may be configured to emit multiple audio signals simultaneously. The audio signals may include one or more anti-noise signals, useful audio signals, or audio signals that are pleasing to the user. By way of example, the acoustic manipulation system 300 may emit anti-noise signals to attenuate noise signals generated by a naturalist while simultaneously or alternatively emitting an audio signal that attracts the type of bird or animal that the naturalist seeks (e.g., a bird call, the sound of a doe in heat, etc.). By way of another example, the acoustic manipulation system 300 may emit anti-noise signals to attenuate noise signals generated by a vehicle traveling along a road while simultaneously playing music. Multiple audio signals may be emitted simultaneously by a single speaker 330, or multiple speakers 330 may be used simultaneously to emit the desired audio signals.

As utilized herein, the terms “approximately,” “about,” “substantially,” and similar terms are intended to have a broad meaning in harmony with the common and accepted usage by those of ordinary skill in the art to which the subject matter of this disclosure pertains. It should be understood by those of skill in the art who review this disclosure that these terms are intended to allow a description of certain features described and claimed without restricting the scope of these features to the precise numerical ranges provided. Accordingly, these terms should be interpreted as indicating that insubstantial or inconsequential modifications or alterations of the subject matter described and claimed are considered to be within the scope of the invention as recited in the appended claims.

It should be noted that the terms “exemplary” and “example” as used herein to describe various embodiments is intended to indicate that such embodiments are possible examples, representations, and/or illustrations of possible embodiments (and such term is not intended to connote that such embodiments are necessarily extraordinary or superlative examples).

The terms “coupled,” “connected,” and the like, as used herein, mean the joining of two members directly or indirectly to one another. Such joining may be stationary (e.g., permanent, etc.) or moveable (e.g., removable, releasable, etc.). Such joining may be achieved with the two members or the two members and any additional intermediate members being integrally formed as a single unitary body with one another or with the two members or the two members and any additional intermediate members being attached to one another.

References herein to the positions of elements (e.g., “top,” “bottom,” “above,” “below,” “between,” etc.) are merely used to describe the orientation of various elements in the figures. It should be noted that the orientation of various elements may differ according to other exemplary embodiments, and that such variations are intended to be encompassed by the present disclosure.

Also, the term “or” is used in its inclusive sense (and not in its exclusive sense) so that when used, for example, to connect a list of elements, the term “or” means one, some, or all of the elements in the list. Conjunctive language such as the phrase “at least one of X, Y, and Z,” unless specifically stated otherwise, is otherwise understood with the context as used in general to convey that an item, term, etc. may be either X, Y, Z, X and Y, X and Z, Y and Z, or X, Y, and Z (i.e., any combination of X, Y, and Z). Thus, such conjunctive language is not generally intended to imply that certain embodiments require at least one of X, at least one of Y, and at least one of Z to each be present, unless otherwise indicated.

It is important to note that the construction and arrangement of the systems as shown in the exemplary embodiments is illustrative only. Although only a few embodiments of the present disclosure have been described in detail, those skilled in the art who review this disclosure will readily appreciate that many modifications are possible (e.g., variations in sizes, dimensions, structures, shapes and proportions of the various elements, values of parameters, mounting arrangements, use of materials, colors, orientations, etc.) without materially departing from the novel teachings and advantages of the subject matter recited. For example, elements shown as integrally formed may be constructed of multiple parts or elements. It should be noted that the elements and/or assemblies of the components described herein may be constructed from any of a wide variety of materials that provide sufficient strength or durability, in any of a wide variety of colors, textures, and combinations. Accordingly, all such modifications are intended to be included within the scope of the present inventions. Other substitutions, modifications, changes, and omissions may be made in the design, operating conditions, and arrangement of the preferred and other exemplary embodiments without departing from scope of the present disclosure or from the spirit of the appended claim.

What is claimed is:

1. An odor neutralizing unit, comprising:
a housing;
an odor neutralizing gas emitter coupled to the housing and configured to provide an odor neutralizing gas that neutralizes odor particles; and
an active noise canceling system, comprising:
a microphone configured to generate a captured signal representing a noise signal;
an acoustic transducer configured to emit audio signals; and
a controller operatively coupled to the microphone and the acoustic transducer and configured to receive the captured signal from the microphone, wherein the controller is configured to control the acoustic transducer to emit an anti-noise signal based on the captured signal, and wherein the anti-noise signal is configured to attenuate the noise signal.

2. The odor neutralizing unit of claim 1, further comprising a fan coupled to the housing, wherein the housing defines an inlet, an outlet, and an internal volume extending between the inlet and the outlet, wherein the fan is configured to draw air into the internal volume through the inlet, wherein the odor neutralizing gas emitter is configured to provide the odor neutralizing gas to the internal volume such that the air mixes with the odor neutralizing gas, and wherein the fan is configured to expel a mixture of the air and the odor neutralizing gas from the internal volume through the outlet.

3. The odor neutralizing unit of claim 2, wherein the odor neutralizing gas emitter generates an emitter noise signal during operation, wherein the fan generates a fan noise signal during operation, and wherein the noise signal includes at least one of the emitter noise signal and the fan noise signal such that the anti-noise signal attenuates the at least one of the emitter noise signal and the fan noise signal.

4. The odor neutralizing unit of claim 2, further comprising a deflector positioned adjacent the outlet, wherein the fan is configured to move the air longitudinally through the internal volume, and wherein the deflector is configured to redirect the mixture of the air and the odor neutralizing gas such that the mixture moves laterally through the outlet.

5. The odor neutralizing unit of claim 1, wherein the noise signal includes at least one outside noise signal originating outside of the scent neutralizing unit such that the anti-noise signal attenuates the at least one outside noise signal.

6. The odor neutralizing unit of claim 1, further comprising:
a fan coupled to the housing and configured to move the odor neutralizing gas away from the unit; and
a motion sensor operatively coupled to the controller and configured to detect motion of an object relative to the housing,
wherein, in response to the motion sensor detecting motion, the controller is configured to vary operation of at least one of the odor neutralizing gas emitter and the fan.

7. The odor neutralizing unit of claim 1, further comprising a fan coupled to the housing and configured to move the odor neutralizing gas away from the unit, wherein the captured signal is a first captured signal, wherein the controller is selectively reconfigurable between a teaching mode and a working mode, wherein during the teaching mode:
the odor neutralizing gas emitter and the fan are disabled;
the microphone generates a second captured signal representing at least one background audio signal; and
the controller records the second captured signal; and
wherein, during the working mode, the controller is configured to control the acoustic transducer to emit the anti-noise signal based on the first captured signal and the second captured signal.

8. The odor neutralizing unit of claim 1, wherein the acoustic transducer is a speaker configured to emit audio signals, and wherein the speaker is selectively repositionable relative to the housing.

9. The odor neutralizing unit of claim 1, further comprising a timekeeping device operatively coupled to the controller and configured to provide time data relating to at least one of a current date, a current time, and a time since an event has occurred, wherein the controller is configured to use the time data to determine whether the controller is operating during a standby time range, and wherein the controller is configured to deactivate the odor neutralizing gas emitter in response to a determination that the controller is operating during the standby time range.

10. The odor neutralizing unit of claim 1, further comprising a resonator defining a resonator volume in fluid communication with a resonator passage, wherein the resonator is configured to emit a second anti-noise signal in response to a movement of gas relative to the resonator passage.

11. The odor neutralizing unit of claim 10, further comprising a fan coupled to the housing and configured to move air through an internal volume defined by the housing, wherein the resonator passage is in fluid communication with the internal volume, wherein the odor neutralizing gas emitter generates an emitter noise signal during operation, wherein the fan generates a fan noise signal during operation, and wherein the second anti-noise signal is configured to attenuate at least one of the emitter noise signal and the fan noise signal.

12. The odor neutralizing unit of claim 1, wherein the microphone is a directional microphone configured to be more sensitive to audio signals traveling toward the directional microphone along a first axis than to audio signals traveling toward the directional microphone along a second axis angularly offset from the first axis.

13. The odor neutralizing unit of claim 1, wherein the odor neutralizing gas emitter is an ozone generator, and wherein the odor neutralizing gas is ozone.

14. An odor neutralizing unit, comprising:
a housing defining an inlet, an outlet, and an internal volume extending between the inlet and the outlet;
an odor neutralizing gas emitter coupled to the housing and configured to provide an odor neutralizing gas that neutralizes odor particles into the internal volume, wherein the odor neutralizing gas emitter generates an emitter noise signal during operation; and
a fan coupled to the housing and configured to draw air into the internal volume through the inlet and expel a mixture of the air and the odor neutralizing gas from the internal volume through the outlet, wherein the fan generates a fan noise signal during operation;
at least one baffle coupled to the housing and extending into the internal volume, wherein the at least one baffle is configured to attenuate at least one of the emitter noise signal and the fan noise signal.

15. The odor neutralizing unit of claim 14, wherein the at least one baffle includes a plurality of baffles, and wherein at least two of the plurality of baffles define a duct therebetween, and wherein the duct is in fluid communication with the inlet.

16. The odor neutralizing unit of claim 15, further comprising an isolator coupling the fan to the housing, wherein the isolator includes a compliant material, and wherein the isolator prevents the fan from directly contacting the housing.

17. The odor neutralizing unit of claim 16, further comprising a resonator defining a resonator volume in fluid communication with a resonator passage, wherein the resonator passage is fluidly coupled to the internal volume, wherein the resonator is configured to emit a second anti-noise signal in response to a movement of gas relative to the resonator passage, and wherein the second anti-noise signal is configured to attenuate at least one of the emitter noise signal and the fan noise signal.

18. An odor neutralizing unit, comprising:
a housing defining an inlet, an outlet, and an internal volume extending between the inlet and the outlet;
an ozone generator extending within the housing and configured to provide ozone gas that neutralizes odor particles;
a fan coupled to the housing and configured to draw air into the internal volume through the inlet and expel a mixture of the air and the ozone gas from the internal volume through the outlet, wherein the fan generates a fan noise signal during operation;
at least one baffle coupled to the housing and extending into the internal volume, wherein the at least one baffle is configured to attenuate the fan noise signal; and
an active noise canceling system, comprising:
a microphone configured to generate a captured signal representing the fan noise signal;
a speaker configured to produce sound; and
a controller operatively coupled to the microphone and the speaker and configured to receive the captured signal from the microphone, wherein the controller is configured to control the speaker to emit an anti-noise signal based on the captured signal, and wherein the anti-noise signal is configured to attenuate the fan noise signal.

19. The odor neutralizing unit of claim 18, wherein the at least one baffle includes a plurality of baffles, and wherein at least two of the plurality of baffles define a duct therebetween, and wherein the duct is in fluid communication with the inlet.

20. The odor neutralizing unit of claim 19, further comprising a deflector positioned adjacent the outlet, wherein the fan is configured to move the air longitudinally through the internal volume, and wherein the deflector is configured to redirect the mixture of the air and the ozone gas such that the mixture moves laterally through the outlet.

21. A method of neutralizing odors, comprising:
providing, by an odor neutralizing gas emitter, an odor neutralizing gas configured to neutralize odor particles into an internal volume of a housing, the housing defining an inlet and an outlet fluidly coupled to the internal volume;
moving, by a fan, air into the internal volume through the inlet;
expelling, by the fan, a mixture of the air and the odor neutralizing gas through the outlet;
generating, by at least one of the odor neutralizing gas emitter and the fan, a noise signal; and
emitting, by an acoustic transducer, an anti-noise signal configured to attenuate the noise signal generated by the at least one of the odor neutralizing gas emitter and the fan.

22. The method of claim 21, further comprising:
observing, by a microphone, the noise signal and generating a captured signal representing the noise signal; and
determining at least one characteristic of the anti-noise signal based on the captured signal.

23. The method of claim 22, wherein a plurality of baffles are coupled to the housing and extend into the internal volume, and wherein at least two of the plurality of baffles define a duct therebetween.
